(19) United States
(12) Patent Application Publication
(10) Pub. No.: US 2024/0173366 A1
(43) Pub. Date: May 30, 2024

(54) AIRWAY MEDICAMENTS

(71) Applicant: Trench Therapeutics, Inc., Portola Valley, CA (US)

(72) Inventors: David S. HARBURGER, Carlisle, MA (US); Prashant GIRINATH, Arlington, MA (US)

(21) Appl. No.: 18/540,958

(22) Filed: Dec. 15, 2023

Related U.S. Application Data
(63) Continuation of application No. PCT/US2022/035547, filed on Jun. 29, 2022.
(60) Provisional application No. 63/216,631, filed on Jun. 30, 2021.

(51) Int. Cl. A61K 35/744; A61K 35/00; A61K 35/741; A61P 31/04

(52) U.S. Cl. CPC A61K 35/744; A61K 35/741; A61P 31/04; A61K 2035/115

(57) ABSTRACT

Described herein are compositions, methods, kits and devices for the treatment and/or prevention upper respiratory tract and peripheral auditory system conditions. In particular, bacterial populations described herein are live, purified bacteria for the modulation, restoration and/or promotion of the microbiome in the upper respiratory tract of a subject, including the nasal cavity, to promote health. Such bacterial populations may include single or multiple strains for bacteria. The multiple strains of bacteria may be strains from the same or different species, including species of Corynebacterium and/or Dolosigranulum pigrum.

### CROSS REFERENCE TO RELATED APPLICATIONS

This application is a continuation of International Application No. PCT/US2022/035547, filed Jun. 29, 2022, which claims the benefit of U.S. Provisional Application No. 63/216,631 filed Jun. 30, 2021, the disclosures of which are incorporated herein by reference in their entirety.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### BACKGROUND

Host and environmental factors influence the integrity of the microbiome in subjects. Microbial imbalance can lead to inflammation (or vice versa), resulting in opportunistic pathogenic microorganism colonization and subsequent disease conditions. Probiotic bacteria provide a therapeutic opportunity for addressing microbiome imbalance and disease related conditions. Typically, probiotic bacteria in clinical development to date have focused on gut colonizing bacteria. In some instances, there may be a need for forms of intervention which promote an improved microbiome environment in the upper respiratory tract, in particular the nasal cavity, for the treatment and prevention of conditions in the nasal cavity as well as in systems linked to the upper respiratory tract by a shared mucosal network, such as the middle ear, which is connected to the nasopharynx through the eustachian tube.

### BRIEF SUMMARY

Provided herein are compositions, including pharmaceutical compositions, methods, kits and devices for modification of microbiome in a subject. As described in more detail herein, such compositions provide for, among other things, defending the integrity of the nasal cavity microbiome, and beneficially imparting downstream effects on the upper respiratory tract (including the nasal cavity) and middle ear for treatment or prevention of ailments of the middle ear. Further provided herein are mixtures of live, purified bacterial strains affording various bactericidal mechanisms to pathogenic bacteria, and in some instances the live, purified bacteria are mutualistic in their relationship to each other. In some embodiments, the live, purified bacteria are selected from Corynebacterium species and, optionally, Dolosigranulum species. In some embodiments, the live, purified bacteria are species of Corynebacterium and/or Dolosigranulum. In some embodiments, the live, purified bacteria comprise a strain listed in Table 1 and/or Table 2. In some embodiments, the Corynebacterium species is C. pseudodiphtheriticum, C. accolens, C. amycolatum, C. propinquum, C. glutamicum, or C. striatum. In some embodiments, the Corynebacterium species is a combination of at least two species selected from C. pseudodiphtheriticum, C. accolens, C. amycolatum, C. propinquum, C. glutamicum, and C. striatum. In some embodiments, the Corynebacterium species is C. pseudodiphtheriticum. In some embodiments, the composition comprises at least two strains of C. pseudodiphtheriticum. In some embodiments, the Corynebacterium species is C. pseudodiphtheriticum and the Dolosigranulum species is D. pigrum. Further provided herein are compositions wherein the live, purified bacteria comprise bacteria having a high percent identity, e.g., at least 97% identity, based on comparison to whole genome, the entire 16S rRNA region, or a hypervariable region of the 16S rRNA (e.g., V4 region), to a bacterial strain listed in Table 1 or Table 2.

Provided here are methods for treatment of a middle ear infection, the method comprising the steps of: administering to a subject a live, purified population of bacteria, wherein the live, purified population of bacteria comprises: a plurality of s strains of Corynebacterium; and optionally, at least one strain of Dolosigranulum pigrum, wherein the live, purified population of bacteria is present in an amount sufficient for treatment of a middle ear infection. Further provided herein are methods, wherein the middle ear infection is otitis media. Further provided herein are methods, wherein the otitis media is acute otitis media, otitis media with effusion, or chronic otitis media with effusion. Further provided herein are methods, wherein the live, purified population of bacteria is further present in an amount sufficient for reduction of ear pain. Further provided herein are methods, wherein the live, purified population of bacteria is further present in an amount sufficient for reduction of fever. Further provided herein are methods, wherein the live, purified population of bacteria is present in an amount sufficient for reduction in colonization of a pathogenic bacteria in a nasal cavity of the subject. Further provided herein are methods, wherein the plurality of strains of Corynebacterium comprises strains of C. accolens, C. pseudodiphtheriticum, C. amycolatum, C. propinquum, C. glutamicum, or C. striatum. Further provided herein are methods, wherein the plurality of strains of Corynebacterium comprises 1, 2, 3, 4, 5 or more strains listed in Table 1. Further provided herein are methods, wherein the live, purified population of bacteria comprises the at least one strain of Dolosigranulum pigrum. Further provided herein are methods, wherein the at least one strain of Dolosigranulum pigrum comprises 1, 2, 3, 4, 5 or more strains listed in Table 2. Further provided herein are methods, wherein the plurality of strains of Corynebacterium comprises a plurality of strains of Corynebacterium pseudodiphtheriticum. Further provided herein are methods, wherein the plurality of strains of Corynebacterium pseudodiphtheriticum comprises JCM 1320 and/or ATCC 10700. Further provided herein are methods, wherein the plurality of strains of Corynebacterium comprises a plurality of strains of C. accolens. Further provided herein are methods, wherein the plurality of strains of C. accolens comprises ATCC 49726 and/or KPL1818. Further provided herein are methods, wherein the plurality of strains of Corynebacterium comprises a plurality of strains of C. amycolatum. Further provided herein are methods, wherein the plurality of strains of C. amycolatum comprises HM-109 and/or DSM1567. Further provided herein are methods, wherein the live, purified population of bacteria is administered intranasally. Further provided herein are methods, wherein the subject is an infant. Further provided herein are methods, wherein the subject is a child. Further provided herein are methods, wherein the subject is an adult. Further provided herein are methods, wherein the live, purified, population of bacteria is present in a total amount of up to 10\hat{ }15 cfu. Further provided herein are methods, wherein the live, purified, population of bacteria is present in a total amount of at least 10\hat{ }3 cfu, optionally 10\hat{ }3 to 10\hat{ }12 cfu.

Provided here are methods for improving auditory perception in a subject, comprising: administering to a subject a live, purified population of bacteria, wherein the live, purified population of bacteria comprises: a plurality of strains of Corynebacterium; and optionally, at least one strain of Dolosigranulum pigrum, wherein the live, purified population of bacteria is present in an amount sufficient for improving auditory perception in the subject. Further provided herein are methods, wherein the subject has a deficit in auditory perception. Further provided herein are methods, wherein the plurality of strains of Corynebacterium comprises strains of C. accolens, C. pseudodiphtheriticum, C. amycolatum, C. propinquum, C. glutamicum, or C. striatum. Further provided herein are methods, wherein the plurality of strains of Corynebacterium comprises 1, 2, 3, 4, 5 or more strains listed in Table 1. Further provided herein are methods, wherein the live, purified population of bacteria comprises the at least one strain of Dolosigranulum pigrum. Further provided herein are methods, wherein the at least one strain of Dolosigranulum pigrum comprises 1, 2, 3, 4, 5 or more strains listed in Table 2. Further provided herein are methods, wherein the plurality of strains of Corynebacterium comprises a plurality of strains of Corynebacterium pseudodiphtheriticum. Further provided herein are methods, wherein the plurality of strains of Corynebacterium pseudodiphtheriticum comprises JCM 1320 and/or ATCC 10700. Further provided herein are methods, wherein the live, purified population of bacteria is administered intranasally. Further provided herein are methods, wherein the subject is an infant. Further provided herein are methods, wherein the subject is a child. Further provided herein are methods, wherein the subject is an adult. Further provided herein are methods, wherein the live, purified, population of bacteria is present in a total amount of up to 10\hat{ }15 cfu. Further provided herein are methods, wherein the live, purified, population of bacteria is present in a total amount of 10\hat{ }3 to 10\hat{ }12 cfu.

Provided herein are methods for reduction of ear swelling, ear redness, ear fluid, or ear mucus, the method comprising the steps of: administering to a subject a live, purified population of bacteria, wherein the live, purified population of bacteria comprises: a plurality of strains of Corynebacterium; and optionally, at least one strain of Dolosigranulum pigrum, wherein the live, purified population of bacteria is present in an amount sufficient for of ear swelling, ear redness, ear fluid, or ear mucus. Further provided herein are methods, wherein the plurality of strains of Corynebacterium comprises strains of C. accolens, C. pseudodiphtheriticum, C. amycolatum, C. propinquum, C. glutamicum, or C. striatum. Further provided herein are methods, wherein the plurality of strains of Corynebacterium comprises 1, 2, 3, 4, 5 or more strains listed in Table 1. Further provided herein are methods, wherein the live, purified population of bacteria comprises the at least one strain of Dolosigranulum pigrum. Further provided herein are methods, wherein the at least one strain of Dolosigranulum pigrum comprises 1, 2, 3, 4, 5 or more strains listed in Table 2. Further provided herein are methods, wherein the plurality of strains of Corynebacterium comprises a plurality of strains of Corynebacterium pseudodiphtheriticum. Further provided herein are methods, wherein the plurality of strains of Corynebacterium pseudodiphtheriticum comprises JCM 1320 and/or ATCC 10700. Further provided herein are methods, wherein the live, purified population of bacteria is administered intranasally. Further provided herein are methods, wherein the subject is an infant. Further provided herein are methods, wherein the subject is a child. Further provided herein are methods, wherein the subject is an adult. Further provided herein are methods, wherein the live, purified, population of bacteria is present in a total amount of up to 10\hat{ }15 cfu. Further provided herein are methods, wherein the live, purified, population of bacteria is present in a total amount of 10\hat{ }3 to 10\hat{ }12 cfu. Further provided herein are methods, wherein the live, purified, population of bacteria is present in an amount sufficient for a reduction in colonization of Streptococcus pneumoniae, Haemophilus influenzae, or Moraxella catarrhalis in the middle ear.

Provided herein are methods for treatment of a middle ear infection, the method comprising the steps of: administering to a subject a live, purified population of bacteria, wherein the live, purified population of bacteria comprises: a plurality of strains of Dolosigranulum pigrum; and optionally, at least one strain of Corynebacterium, wherein the live, purified population of bacteria is present in an amount sufficient for treatment of a middle ear infection. Further provided herein are methods, wherein the middle ear infection is otitis media. Further provided herein are methods, wherein the otitis media is acute otitis media, otitis media with effusion, or chronic otitis media with effusion. Further provided herein are methods, wherein the live, purified population of bacteria is further present in an amount sufficient for reduction of ear pain. Further provided herein are methods, wherein the live, purified population of bacteria is further present in an amount sufficient for reduction of fever. Further provided herein are methods, wherein the live, purified population of bacteria is present in an amount sufficient for reduction in colonization of a pathogenic bacteria in a nasal cavity of the subject. Further provided herein are methods, wherein the live, purified population of bacteria comprises the at least one strain of Corynebacterium. Further provided herein are methods, wherein the at least one of strain of Corynebacterium comprises a strain of C. accolens, C. pseudodiphtheriticum, C. amycolatum, C. propinquum, C. glutamicum, or C. striatum. Further provided herein are methods, wherein the at least one of strain of Corynebacterium comprises 1, 2, 3, 4, 5 or more strains listed in Table 1. Further provided herein are methods, wherein the plurality of strains of Dolosigranulum pigrum comprises 1, 2, 3, 4, 5 or more strains listed in Table 2. Further provided herein are methods, wherein the at least one of strain of Corynebacterium comprises a plurality of strains of Corynebacterium pseudodiphtheriticum. Further provided herein are methods, wherein the at least one of strain of Corynebacterium comprises JCM 1320 and/or ATCC 10700. Further provided herein are methods, wherein the live, purified population of bacteria is administered intranasally. Further provided herein are methods, wherein the subject is an infant. Further provided herein are methods, wherein the subject is a child. Further provided herein are methods, wherein the subject is an adult. Further provided herein are methods, wherein the live, purified, population of bacteria is present in a total amount of up to 10\hat{ }15 cfu. Further provided herein are methods, wherein the live, purified, population of bacteria is present in a total amount of at least 10\hat{ }3 cfu, optionally 10\hat{ }3 to 10\hat{ }12 cfu. Further provided herein are methods, wherein the live, purified, population of bacteria is present in an amount sufficient for a reduction in colonization of Streptococcus pneumoniae, Haemophilus influenzae, or Moraxella catarrhalis in the middle ear.

Provided herein are methods for improving auditory perception in a subject, comprising: administering to a subject a live, purified population of bacteria, wherein the live, purified population of bacteria comprises: a plurality of strains of Dolosigranulum pigrum; and optionally, at least one strain of Corynebacterium, wherein the live, purified population of bacteria is present in an amount sufficient for improving auditory perception in the subject. Further provided herein are methods, wherein the subject has a deficit in auditory perception. Further provided herein are methods, wherein the live, purified population of bacteria comprises the at least one strain of Corynebacterium. Further provided herein are methods, wherein the at least one of strain of Corynebacterium comprises a strain of C. accolens, C. pseudodiphtheriticum, C. amycolatum, C. propinquum, C. glutamicum, or C. striatum. Further provided herein are methods, wherein the at least one of strain of Corynebacterium comprises 1, 2, 3, 4, 5 or more strains listed in Table 1. Further provided herein are methods, wherein the plurality of strains of Dolosigramilum pigrum comprises 1, 2, 3, 4, 5 or more strains listed in Table 2. Further provided herein are methods, wherein the at least one of strain of Corynebacterium comprises a plurality of strains of Corynebacterium pseudodiphtheriticum. Further provided herein are methods, wherein the at least one of strain of Corynebacterium comprises JCM 1320 and/or ATCC 10700. Further provided herein are methods, wherein the live, purified population of bacteria is administered intranasally. Further provided herein are methods, wherein the subject is an infant. Further provided herein are methods, wherein the subject is a child. Further provided herein are methods, wherein the subject is an adult. Further provided herein are methods, wherein the live, purified, population of bacteria is present in a total amount of up to 10\hat{ }15 cfu. Further provided herein are methods, wherein the live, purified, population of bacteria is present in a total amount of at least 10\hat{ }3 cfu, optionally 10\hat{ }3 to 10\hat{ }12 cfu. Further provided herein are methods, wherein the live, purified, population of bacteria is present in an amount sufficient for a reduction in colonization of Streptococcus pneumoniae, Haemophilus influenzae, or Moraxella catarrhalis in the middle ear.

Provided herein are methods for reduction of ear swelling, ear redness, ear fluid, or ear mucus, the method comprising the steps of: administering to a subject a live, purified population of bacteria, wherein the live, purified population of bacteria comprises: a plurality of strains of Dolosigranulum pigrum; and optionally, at least one strain of Corynebacterium, wherein the live, purified population of bacteria is present in an amount sufficient for of ear swelling, ear redness, ear fluid, or ear mucus. Further provided herein are methods, wherein the live, purified population of bacteria comprises the at least one strain of Corynebacterium. Further provided herein are methods, wherein the at least one of strain of Corynebacterium comprises a strain of C. accolens, C. pseudodiphtheriticum, C. amycolatum, C. propinquum, C. glutamicum, or C. striatum. Further provided herein are methods, wherein the at least one of strain of Corynebacterium comprises 1, 2, 3, 4, 5 or more strains listed in Table 1. Further provided herein are methods, wherein the plurality of strains of Dolosigranulum pigrum comprises 1, 2, 3, 4, 5 or more strains listed in Table 2. Further provided herein are methods, wherein the at least one of strain of Corynebacterium comprises a plurality of strains of Corynebacterium pseudodiphtheriticum. Further provided herein are methods, wherein the at least one of strain of Corynebacterium comprises JCM 1320 and/or ATCC 10700. Further provided herein are methods, wherein the live, purified population of bacteria is administered intranasally. Further provided herein are methods, wherein the subject is an infant. Further provided herein are methods, wherein the subject is a child. Further provided herein are methods, wherein the subject is an adult. Further provided herein are methods, wherein the live, purified, population of bacteria is present in a total amount of up to 10\hat{ }15 cfu. Further provided herein are methods, wherein the live, purified, population of bacteria is present in a total amount of at least 10\hat{ }3 cfu, optionally 10\hat{ }3 to 10\hat{ }12 cfu. Further provided herein are methods, wherein the live, purified, population of bacteria is present in an amount sufficient for a reduction in colonization of Streptococcus pneumoniae, Haemophilus influenzae, or Moraxella catarrhalis in the middle ear.

### BRIEF DESCRIPTION OF THE DRAWINGS

FIG. 1: Illustrates, in some embodiments, various dosage forms described herein for oral 101 and/or intranasal 102 administration. Dosage forms illustrated include an inhaler 103 for aerosol administration, liquid 104 and capsule 105 for oral administration, and a spray bottle 106 for intranasal administration.

FIG. 2A, in some embodiments, illustrates a plot of OD600 measurements from growth of ATCC 10700, with OD600 for the Y axis and time in hours on the X axis.

FIG. 2B, in some embodiments, illustrates a plot of OD600 measurements from growth of ATCC 10700, with Log(OD600) for the Y axis and time in hours on the X axis.

FIG. 3A, in some embodiments, illustrates a plot of OD600 measurements from growth of JCM 1320, with OD600 for the Y axis and time in hours on the X axis.

FIG. 3B, in some embodiments, illustrates a plot of OD600 measurements from growth of JCM 1320, with Log(OD600) for the Y axis and time in hours on the X axis.

FIG. 4A, in some embodiments, illustrates a plot of OD600 measurements from growth of cultures having varying amounts of ATCC 10700 and JCM 1320, with OD600 for the Y axis and time in hours on the X axis.

FIG. 4B, in some embodiments, illustrates a plot of OD600 measurements from growth of cultures having varying amounts of ATCC 10700 and JCM 1320, with Log(OD600) for the Y axis and time in hours on the X axis.

FIG. 5, in some embodiments, is an image capture of an agar plate, showing four spottings of ATCC 10700 on the left, and four spottings of JCM 1320 on the right. The image capture was taken after 24 hours of growth.

FIG. 6, in some embodiments, is an image capture of an agar plate, showing columns of left to right with two spottings of ATCC 10700, two spottings of D. pigrum, two spottings of D. pigrum, and two spottings of JCM 1320. The image capture was taken after 24 hours of growth.

### DETAILED DESCRIPTION

Provided herein are composition, methods, kits and devices relating to upper respiratory tract colonizing bacteria for prevention and/or treatment of respiratory tract conditions and/or middle ear conditions. Furthermore, provided herein are (1) probiotic bacterial mixtures (2) excipients, dosage forms and routes of administration for such mixtures, (3) and conditions for treatment with such probiotic bacterial mixtures.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included, unless the context clearly dictates otherwise.

The terminology used herein is for the purpose of describing particular instances only and is not intended to be limiting of any embodiment. As used herein, the singular forms “a,” “an” and “the” are intended to include the plural forms as well, unless the context clearly indicates otherwise. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The term “subject” as used herein includes human and non-human mammals, including for example: a primate, cow, horse, pig, sheep, goat, dog, cat, or rodent, capable of being colonized by other organisms.

In some embodiments, provided herein are compositions which include bacteria having a percent identity based on 16S rRNA bacterial genetic sequence, a hypervariable region of the 16S rRNA, or whole genome comparison to a reference strain. Typically, comparison of the 16S rRNA bacterial genetic sequence allows a strain to be identified as within the same species as another strain by comparing sequences with known bacterial DNA sequences using NCBI BLAST search. The level of identity in relation to a nucleotide sequence may be determined for at least 20 contiguous nucleotides, for at least 30 contiguous nucleotides, for at least at least 40 contiguous nucleotides, for at least 50 contiguous nucleotides, for at least 60 contiguous nucleotides, or for at least 100 contiguous nucleotides. In some embodiments, the level of identity in relation to a nucleotide sequence is determined for the entire sequence searched. Percent identity may be at least 70%, 80%, 85%, 90%, 91%, 92%, 93%, 94%, 95%, 96%, 97%, 98%, 99%, or 100% to a reference bacterial 16S rRNA sequence, 16S rRNA V4 region sequence, or whole genome sequence. Percent identity may be at least 70%, 80%, 85%, 90%, 91%, 92%, 93%, 94%, 95%, 96%, 97%, 98%, 99%, or 100% to a reference bacteria 16S rRNA: V1 region, V2 region, V3 region, V5 region, V6 region, V7 region, V8 region or V9 region sequence.

Provided herein are compositions comprising live bacteria. Various techniques for determining if the bacteria is live include measuring membrane stability, transcription, translation, and cell division. In some embodiments, a live bacterium can comprise a bacterium that retains membrane stability. In some embodiments, a live bacterium can comprise a bacterium that is capable of transcription and translation. In some embodiments, a live bacterium can comprise a bacterium that is capable of cell division. In some embodiments, live bacteria can be determined by a culture dependent or a culture independent technique. In some cases, live bacteria can comprise an individual or a group of bacteria that can produce a colony-forming unit (cfu) when plated on stable growth media. In some embodiments, live and/or dead bacteria can be determined by imaging, for example with a live/dead stain. In some embodiments, a viability PCR based method can be used to determine live bacteria. In some cases, a metabolomic assay can be used to determine live bacteria.

In some embodiments, reference to a population of bacteria or a purified population refers to a plurality of bacteria. In some embodiments, a purified bacteria is enriched from a source sample. Compositions described herein can comprise about: 10%, 20%, 30%, 40%, 50%, 60%, 70% or more of a single strain of bacteria.

As used herein, a substance is “pure” or “substantially pure” if it is substantially free of other components. The terms “purify,” “purifying” and “purified”, when applied to a bacterium, can refer to a bacterium that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, or by passage through culture, and a purified bacterium or bacterial population may contain other materials up to at least about 10%, about 20%, about 30%, about 40%, about 50%, about 60%, about 70%, about 80%, about 90%, or above about 90% and still be considered “isolated.” Purified bacteria and bacterial populations can be more than at least about 80%, about 85%, about 90%, about 91%, about 92%, about 93%, about 94%, about 95%, about 96%, about 97%, about 98%, about 99%, or more than at least about 99% pure by weight (w/w). In the instance of microbial compositions provided herein, the one or more bacterial types (species or strains) present in the composition can be independently purified from one or more other bacteria produced and/or present in the material or environment containing the bacterial type. Microbial compositions and the bacterial components thereof are generally purified from residual habitat products.

An isolated bacterium may have been (1) separated from at least some of the components with which it was associated when initially obtained (whether in nature or in an experimental setting), and/or (2) produced, prepared, purified, and/or manufactured by the hand of man, e.g. using artificial culture conditions such as (but not limited to) culturing on a plate and/or in a fermenter. Isolated bacteria can include those bacteria that are cultured, even if such cultures are not monocultures. Isolated bacteria can be separated from at least about 10%, about 20%, about 30%, about 40%, about 50%, about 60%, about 70%, about 80%, about 90%, or more of the other components with which they were initially associated. Isolated bacteria can be more than about 80%, about 85%, about 90%, about 91%, about 92%, about 93%, about 94%, about 95%, about 96%, about 97%, about 98%, about 99%, or more than about 99% pure. A bacterial population of a biological sample provided herein can comprise one or more bacteria, which may then be isolated from such sample. Isolated bacteria may be provided in a form that is not naturally occurring.

### 1) Probiotic Bacterial Mixtures

The nasal cavity of the upper respiratory tract is a nutrient-poor, high-salinity niche where bacteria compete for limited resources. A healthy nasal microbiome prevents pathogenic microorganisms from colonization, harmful products from such colonization, inflammation, and generation of “leaky” cell-cell junctions, all of which can result in subsequent disease conditions in other organs along a shared mucosal network, including the peripheral auditory system—the middle ear in particular. The nasal cavity provides an opportunity for imparting beneficial microbiome change to prevent and/or treat many disease conditions. For example, in a small human trial, a beneficial strain of Corynebacterium pseudodiphtheriticum (C. pseudodiphtheriticum strain “090104”) has been reported to show efficacy in elimination of Staphylococcus aureus from the nasal cavity in volunteers exposed to abnormal microclimate and altered gaseous environment, Kiryukhina et al. Probiotics and Antimicrobial Proteins (2013). Nasal spray application of the strain eradicated S. aureus in three subjects and reduced its presence in a methicillin-resistant S. aureus (MRSA) carrier.

Bacteria described herein are to be used to control, treat, reduce, eliminate, and/or prevent pathogenic colonization by an organism on a subject and/or reduce inflammation. In some embodiments, provided herein are compositions for reducing prolonged inflammation. Compositions described herein may be administered or designed for delivery to particular locations of the subject, in particular, the upper respiratory tract, including the anterior nares, nasal cavity, and/or nasopharynx. In some embodiments, compositions described herein colonize in the middle ear. In some embodiments, compositions described herein result in modification of the bacterial constituents of the middle ear. Bacterial strains described herein may be isolated from the upper respiratory tract regions including, without limitation, anterior nares, nasal cavity, and/or nasopharynx.

In some embodiments, provided herein are compositions having bacterial populations having one or more species, and one or more strains for each of the one or more species. In some instances, a composition described herein includes 1, 2, 3, 4, 5, 6, 7, 8, 9, 10 or more species of bacteria. In some instances, a composition described herein includes 1, 2, 3, 4, 5, 6, 7, 8, 9, 10 or more strains of bacteria. Further provided herein are bacterial populations comprising at least one species of Corynebacterium. Further provided herein are bacterial populations comprising a plurality of species of Corynebacterium. Corynebacterium are gram-stain-positive bacteria, non-spore forming and nonmotile. Exemplary Corynebacterium species for inclusion in compositions described herein include: C. accolens, C. afermentans, C. ammoniagenes, C. amycolatum, C. argentoratense, C. aquaticum, C. auris, C. bovis, C. diphtheria, C. equi (now Rhodococcus equi), C. efficiens, C. flavescens, C. glucuronolyticum, C. glutamicum, C. granulosum, C. haemolyticum, C. halofytica, C. kroppenstedtii, C. jeikeium, C. macginleyi, C. matruchotii, C. minutissimum, C. parvum (Propionibacterium acnes), C. paurometabolum, C. propinquum, C. pseudodiphtheriticum (C. hofmannii), C. pseudotuberculosis, C. ovis, C. pyogenes—Trueperella pyogenes, C. urealyticum, C. renale, C. spec, C. striatum, C. temuis, C. ulcerans, C. urealyticum, and C. xerosis. In some instances, a composition described herein comprises 1, 2, 3, 4, 5, 6, 7, 8, 9, 10 of the Corynebacterium strains listed in Table 1. In some embodiments, a population of bacteria described herein comprises a Corynebacterium strain having a 16S rRNA sequence of at least 90, 91, 92, 93, 94, 95, 96, 97, 98, 99, or 100% identity to that of a strain listed in Table 1. In some embodiments, a population of bacteria described herein comprises a Corynebacterium strain having a 16S rRNA sequence of at least 97% sequence identity with that of a strain listed in Table 1. The sequence identity may be based on a 16s rRNA sequence, 16s rRNA hypervariable region sequence, such as V4, or whole genome comparison. The population of bacteria may be part of a pharmaceutical composition. The bacteria may be live and purified. In some embodiments, a composition herein comprises a plurality of strains of C. accolens. In some cases, a plurality of strains of C. accolens comprises ATCC 49726, KPL1818, ATCC 49725, or a combination of any of these. In some embodiments, a composition herein comprises a plurality of strains of C. amycolatum. In some cases, a plurality of strains of C. amycolatum comprises HM-109, DSM1567, DSM6922, or a combination of any of these.

TABLE 1
Corynebacterium strains

| Species | Strain name | Reference or Deposit |
|---|---|---|
| C. pseudodiphtheriticum | KPL1989 | GenBank: AXLR01000000 |
| C. pseudodiphtheriticum | DSM44287 | GenBank: GCA_000688415.1 |
| | | ATCC 10700 |
| C. pseudodiphtheriticum | 090104 | GenBank: AVFF01000000 |
| | | JCM 1320 |
| C. accolens | KPL1818 | GenBank: AXMA01000001.1 |
| C. accolens | DSM 44278 | ATCC 49725 |
| C. accolens | CNCTC Th 4/63 | ATCC 49726 |
| C. amycolatum | DSM6922 | ATCC 49368 |
| C. amycolatum | DSM1567 | MN175937 |
| C. amycolatum | SK46 | HM-109 |
| | | World wide web |
| | | beiresources.org/Catalog/Bacteria/HM- |
| | | 109.aspx |
| C. propinquum | DSM44285 | GCA_000375525.1 |
| C. glutamicum | DSM20300 | ATCC 13032 |
| C. striatum | DSM20668 | ATCC 6940 |

In some embodiments, provided herein are bacterial populations comprising at least one species of Dolosigranulum. Further provided herein are bacterial populations comprising a plurality of species of Dolosigramlum. In some embodiments, provided herein are populations of bacteria comprising at least one strain of Dolosigranulum pigrum. In some instances, a composition described herein comprises 1, 2, 3, 4, 5, 6, 7, 8, 9, 10 of the D. pigrum strains listed in Table 2. In some embodiments, a population of bacteria described herein comprises a D. pigrum strain having a 16S rRNA sequence of at least 90, 91, 92, 93, 94, 95, 96, 97, 98, 99, or 100% sequence identity to that of a strain listed in Table 2. In some embodiments, a population of bacteria described herein comprises a D. pigrum strain having a 16S rRNA sequence of at least 97% sequence identity with that of a strain listed in Table 2. The sequence identity may be based on a 16s rRNA sequence, 16s rRNA hypervariable region sequence, such as V4, or whole genome comparison. The population of bacteria may be part of a pharmaceutical composition. The bacteria may be live and purified.

TABLE 2
Dolosigranulum strains

| Species | Strain name | Reference or Deposit |
|---|---|---|
| D. pigrum | KLP1914 | Brugger et al., BioRxiv (2019). |
| D. pigrum | CDC 39-95 | LaClaire and Facklam, 2000 |
| D. pigrum | CDC 2949-98 | LaClaire and Facklam, 2000 |
| D. pigrum | CDC 4294-98 | LaClaire and Facklam, 2000 |
| D. pigrum | CDC 4420-98 | LaClaire and Facklam, 2000 |
| D. pigrum | CDC 4545-98 | LaClaire and Facklam, 2000 |
| D. pigrum | CDC 4709-98 | LaClaire and Facklam, 2000 |
| D. pigrum | CDC 4199-99 | LaClaire and Facklam, 2000 |
| D. pigrum | CDC 4791-99 | LaClaire and Facklam, 2000 |
| D. pigrum | CDC 4792-99 | LaClaire and Facklam, 2000 |
| D. pigrum | AMBR11 | LMG P-31 124 |
| | | 16S rRNA disclosed in |
| | | WO2020128022 |
| D. pigrum | AMBR12 | LMG P-31 154 |
| | | 16S rRNA disclosed in |
| | | WO2020128022 |

Further provided herein are populations of bacteria for colonization to the upper respiratory tract having a combination of strains including strains, from different species. In some embodiments, the populations of bacteria comprise at least one strain of Corynebacterium, and at least one strain of Dolosigramilum. In some embodiments, the populations of bacteria comprise at least one strain of C. pseudodiphtheriticum and at least one strain of D. pigrum. In some embodiments, the populations of bacteria comprise at least one strain listed in Table 1, and at least one strain listed in Table 2. In some embodiments, the populations of bacteria comprise at least one strain having a 16S rRNA sequence of at least 97% sequence identity with that of a strain listed in Table 1, and at least one strain having a 16S rRNA sequence of at least 97% sequence identity with that of a strain listed in Table 2. In further embodiments, 1, 2, 3 or more of the Corynebacterium strains are C. pseudodiphtheriticum strains. The population of bacteria may be part of a pharmaceutical composition. The bacteria may be live and purified. Compositions described herein may have mixtures of species. The mixtures may be up to 1, 2, 3, 4, 5, 6, 7, 8, 9, 10 or more species and may include species listed in Table 1 and/or Table 2. Such species may be present in equal amounts or varied amounts. In some embodiments, each different species is present in at least 1%, 10%, 15%, 20%, 25%, 30%, 35%, 40%, 45%, 50%, or 100% of the total colony forming units (CFUs) for the total CFUs for the population of bacteria. Compositions described herein may have mixtures of strains within a species. The mixtures may be up to 1, 2, 3, 4, 5, 6, 7, 8, 9, 10 or more strains and may be from a species listed in Table 1 and/or Table 2. Such strains may be present in equal amounts or varied amounts. In some embodiments, different strains are present in at least 10%, 15%, 20%, 25%, 30%, 35%, 40%, 45%, 50%, or 100% of the total colony forming units (CFUs) for the total CFUs for the population of bacteria.

In some embodiments, provided herein are bacterial populations comprising at least one species of Lactobacillus. Further provided herein are bacterial populations comprising a plurality of species of Lactobacillus. In some embodiments, a composition herein comprises one or more of the following species of Lactobacillus: L. casei, L. plantarum, L. gasseri, L. crispatus, L. acidophilus, L. jensenii, L. fermentum, L. rhamnosus, L. pentosus, or L. sakei. In some embodiments, a composition herein comprises one or more strains of the following species of Lactobacillus: L. casei, L. plantarum, L. gasseri, L. crispatus, L. acidophilus, L. jensenii, L. fermentum, L. rhamnosus, L. pentosus, L. plantarum, or L. sakei.

In some instances, a composition described herein comprises 1, 2, 3, 4, 5, 6, 7, 8, 9, 10 of the Lactobacillus strains listed in Table 3. In some embodiments, a population of bacteria described herein comprises a Lactobacillus strain having a 16S rRNA sequence of at least 90, 91, 92, 93, 94, 95, 96, 97, 98, 99, or 100% sequence identity to that of a strain listed in Table 3. In some embodiments, a population of bacteria described herein comprises a Lactobacillus strain having a 16S rRNA sequence of at least 97% sequence identity with that of a strain listed in Table 3. The sequence identity may be based on a 16s rRNA sequence, a 16s rRNA hypervariable region sequence, such as V4, or whole genome comparison. The population of bacteria may be part of a pharmaceutical composition. The bacteria may be live and purified. Compositions described herein may have mixtures of species. The mixtures may be up to 1, 2, 3, 4, 5, 6, 7, 8, 9, 10 or more species and may include species listed in Table 1, Table 2, Table 3, or any combination thereof. Such species may be present in equal amounts or varied amounts. In some embodiments, each different species is present in at least 1%, 10%, 15%, 20%, 25%, 30%, 35%, 40%, 45%, 50%, or 100% of the total colony forming units (CFUs) for the total CFUs for the population of bacteria. Compositions described herein may have mixtures of strains within a species. The mixtures may be up to 1, 2, 3, 4, 5, 6, 7, 8, 9, 10 or more strains and may be from a species listed in Table 1, Table 2, Table 3, or any combination thereof. Such strains may be present in equal amounts or varied amounts. In some embodiments, different strains are present in at least 10%, 15%, 20%, 25%, 30%, 35%, 40%, 45%, 50%, or 100% of the total colony forming units (CFUs) for the total CFUs for the population of bacteria.

TABLE 3
Lactobacillus strains

| Species | Strain name | Reference and/or Deposit |
|---|---|---|
| L. casei | AMBR2 | De Boeck et al., Cell reports |
| | | (2020). Belgian Co-ordinated |
| | | Collection of Micro-Organisms |
| | | Deposit LMG P-30039 |
| | | Lab of Applied Microbiology |
| | | and Biotechnology (LAMB) |
| | | PRJEB21025 |
| L. sakei | AMBR8 | De Boeck et al., Cell reports |
| | | (2020). LAMB PRJEB32716 |
| L. rhamnosus | GG | De Boeck et al., Cell reports |
| | | (2020). ATCC 53103 |

Corynebacteria are Gram-positive, non-motile, facultative anaerobes, characterized as having the appearance of straight or slightly curved slender rods with tapered or clubbed ends. Corynebacterium pseudodiphtheriticum, previously designated as Corynebacterium hofmannii, is a nonlipophilic, nonfermentive, urease- and nitrate-positive Corynebacterium species, which is part of the oropharyngeal bacterial flora. Corynebacterium accolens are Gram positive rods, irregularly shaped (‘coryneforms”), arranged as single cells, in pairs, in V forms, in palisades, or in clusters. Corynebacterium amycolatum, are Gram positive rods, irregularly shaped (‘coryneforms”), they are arranged as single cells, in pairs, in V forms, in palisades, or in clusters. Dolosigranulum pigrum is gram-positive, coccus arranged in pairs, tetrads, and clusters. Lactobacillus is a genus of Gram-positive, aerotolerant anaerobes or microaerophilic, rod-shaped, non-spore-forming bacteria.

In some embodiments, when administered to a subject, bacterial populations described herein reduce or eliminate colonization in the respiratory tract and/or middle ear of pathogenic bacteria. In some embodiments, when administered to a subject, bacterial populations described herein reduce abundance in the respiratory tract and/or middle ear of pathogenic bacteria. Exemplary respiratory tract pathogenic bacteria include, without limitation, Staphylococcus aureus, Streptococcus pneumoniae, Pseudomonas aeruginosa, and Burkholderia pseudomallei. Exemplary strains of such pathogenic bacteria are listed in Table 4. Such reduction of pathogenic bacteria may be in the upper respiratory tract, the middle ear, or the lower respiratory tract.

TABLE 4
Pathogenic bacteria

| Species | Strain name | Reference |
|---|---|---|
| S. aureus (MRSA) | JE2 | BEI Resources |
| | | NR-46543 |
| S. aureus (MRSA) | LAC | ATCC BAA-1556 |
| S. aureus (MRSA) | Mu50 | ATCC 700699 |
| S. aureus (MSSA) | 96:308/USA900 | ATCC BAA-1749 |
| S. pneumoniae | TIGR4 | ATCC BAA-33 |
| S. pneumoniae | M270-8 | ATCC BAA-1659 |
| S. pneumoniae | DBL5 | AF071810 |
| Pseudomonas aeruginosa | | ATCC 27853 |
| Burkholderia pseudomallei | MSHR520 | GenBank: |
| | | GCA_000583835.1 |
| Streptococcus pneumoniae | (Klein) Chester | ATCC BAA-255 |
| Haemophilus influenzae | Lehmann and | ATCC 19418 |
| | Neumann | |
| Moraxella catarrhalis | (Frosch and Kolle) | ATCC 23246 |
| | Bovre | |

### 2) Excipients, Dosage Forms and Routes of Administrations

To facilitate administration, pharmaceutical compositions described herein may include one or more pharmaceutically acceptable excipients. Example pharmaceutically acceptable excipients include, without limitation, diluents, adjuvants, excipients, water, oils (including petroleum, animal, vegetable or synthetic oils.). Further examples include saline, gum acacia, gelatin, starch paste, talc, keratin, colloidal silica, and urea. Such excipients may include binders such as ethyl cellulose, carboxymethylcellulose, microcrystalline cellulose, or gelatin; excipients such as starch, lactose or dextrins; disintegrating agents such as alginic acid, sodium alginate, Primogel, and cornstarch; lubricants such as magnesium stearate or Sterotex; glidants such as colloidal silicon dioxide; sweetening agents such as sucrose or saccharin, a flavoring agent such as peppermint, methyl salicylate or orange flavoring, or coloring agents. Further examples of excipients include polyethylene glycol, cyclodextrin, oils, or any other similar liquid carrier that may be formulated into a capsule. Still further examples of excipients include sterile diluents such as water, saline solution, physiological saline, Ringer's solution, isotonic sodium chloride, fixed oils such as synthetic mono or digylcerides, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose, thickening agents, lubricating agents, and coloring agents. In some embodiments of the invention, the pharmaceutically acceptable carrier can comprise a growth medium that can support the growth and/or static existence of beneficial bacteria described herein in the context of the pharmaceutical composition prior to administration of the pharmaceutical composition to the subject.

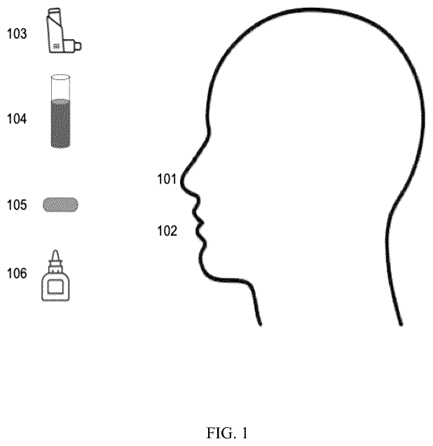

In some instances, a pharmaceutical composition described herein includes materials capable of modifying the physical form of a dosage unit. For example, various dosage forms described herein are illustrated in FIG. 1 for oral 101 and/or intranasal 102 administration. Dosage forms for compositions described herein include a nebulizer or inhaler 103 for aerosol administration, liquid 104 and capsule 105 for oral administration, and a spray bottle 106 for intranasal administration. In some instances, a pharmaceutical composition described herein is located within a nasal spray bottle. In some instances, a pharmaceutical composition described herein is prepared as an aerosol. Aerosols encompass a variety of systems including colloids and pressurized packages. Delivery of a composition in this form may include propulsion of a pharmaceutical composition including the beneficial bacteria described herein through use of liquefied gas or other compressed gas or by a suitable pump system. Aerosols may be delivered in single phase, bi-phasic, or triphasic systems. Compositions, including pharmaceutical compositions, described herein may be formulated depending on the route of administration. Such forms include, without limitation, solutions, suspensions, emulsions, cream, gel, lotion, ointment, tablets, tabs, films, pills, pellets, capsules, capsules including liquids, powders, sustained-release formulations, directed release formulations, lyophylates (generated by freeze drying), emulsions, aerosols, sprays, granules, powders, or syrups. Dosage forms may be, without limitation, liquid, a solid, semisolid, gel, or aerosol. Methods of administration include, but are not limited to, oral, intranasal, or by inhalation. Pharmaceutical compositions described herein may include kits where bacteria described herein are included in a first container (e.g., lyophilized cells), and one or more pharmaceutical acceptable excipients are included in a second container (e.g., water). In some embodiments, provided herein are kits, wherein the kit comprises: a first container, wherein the first container comprises live, purified, and lyophilized population of bacteria that comprises: a plurality of strains of Corynebacterium pseudodiphtheriticum; and a second container, wherein the second container comprises a pharmaceutically acceptable excipient. In some embodiments, provided herein are kits, wherein the kit comprises: a first container, wherein the first container comprises live, purified, and lyophilized population of bacteria that comprises: a plurality of species of Corynebacterium; and a second container, wherein the second container comprises a pharmaceutically acceptable excipient. In some embodiments, provided herein are kits, wherein the kit comprises: a first container, wherein the first container comprises live, purified, and lyophilized population of bacteria that comprises: a plurality of strains of Dolosigramilum pigrum; and a second container, wherein the second container comprises a pharmaceutically acceptable excipient. In some embodiments, provided herein are kits, wherein the kit comprises: a first container, wherein the first container comprises live, purified, and lyophilized population of bacteria that comprises: a plurality of strains of Corynebacterium pseudodiphtheriticum; and a plurality of strains of Dolosigranulum pigrum and; and a second container, wherein the second container comprises a pharmaceutically acceptable excipient. In some embodiments, provided herein are kits, wherein the kit comprises: a first container, wherein the first container comprises live, purified, and lyophilized population of bacteria present in a total amount of at least 10\hat{ }3 cfu that comprises: a strain of Corynebacterium pseudodiphtheriticum; and a strain of Dolosigranulum pigrum; and a second container, wherein the second container comprises a pharmaceutically acceptable excipient. Further provided herein are kits, wherein the live, purified, and lyophilized population of bacteria is present in a total amount of up to 10\hat{ }15 cfu. Further provided herein are kits, wherein the live, purified, and lyophilized population of bacteria is present in a total amount of 10\hat{ }3 to 10\hat{ }12 cfu.

Dosing may include single or multiple administrations of pharmaceutical compositions described herein. Examples include: multiple times a day, daily, every other day, 1, 2, 3, 5, 6, or 7 times a week, weekly, or less often, a single administration, a course of treatment involving several treatments on a regular or irregular basis, or multiple administrations for a period of time until a diminution of colonization is achieved. In some cases, dosing can occur every day, 2 days, 3 days, 4 days, 5 days, 6 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 1 year, 2 years, or as needed. The dosing regimen, including the regularity of and mode of administration, may be dependent on factors including but not limited to the subject being treated; the severity of the condition; the manner of administration, the stage of colonization, the presence of one or more other conditions such as pregnancy, infancy, or the presence of one or more additional diseases. In some embodiments, the subject is an infant. The infant can be up to 24 months old. In some embodiments, the subject is a child. The child may be 2 years to 21 years old. In some embodiments, the subject is an adult. Adults may be 21 years old or more. In some embodiments, the adult is of advanced age, such as 65 years or older.

Compositions, including pharmaceutical compositions, described herein may comprise a single (unit) dose of bacteria. Compositions described herein may comprise about 10² to about 10¹⁵ colony forming units (cfu) of bacteria or a bacterial strain described herein. Compositions described herein may comprise about: 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, about 10⁵ to about 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria or a bacterial strain described herein. In some embodiments, compositions comprise about 10³ cfu, about 10⁴ cfu, about 10⁵ cfu, about 10⁶ cfu, about 10⁷ cfu, about 10⁸ cfu, about 10⁹ cfu, about 10¹⁰ cfu, about 10¹¹ cfu, or about 10¹² cfu of bacteria or a bacterial strain described herein.

Compositions, including pharmaceutical compositions, described herein may comprise 10² to 10¹⁵ colony forming units (cfu) of bacteria or a bacterial strain described herein per mL. Compositions described herein may comprise about 10² to 10¹² cfu, 10³ to 10¹² cfu, 10³ to 10¹¹ cfu, 10³ to 10¹⁰ cfu, 10³ to 10⁹ cfu, 10³ to 10⁸ cfu, 10³ to 10⁷ cfu, 10³ to 10⁶ cfu, 10³ to about 10⁵ cfu, 10³ to 10⁴ cfu, 10⁴ to 10¹² cfu, 10⁴ to 10¹¹ cfu, 10⁴ to 10¹⁰ cfu, 10⁴ to 10⁹ cfu, 10⁴ to 10⁸ cfu, 10⁴ to 10⁷ cfu, 10⁴ to 10⁶ cfu, 10⁵ to 10¹² cfu, 10⁵ to 10¹¹ cfu, about 10⁵ to about 10¹⁰ cfu, 10⁶ to 10¹² cfu, 10⁷ to 10¹² cfu, 10⁸ to 10¹² cfu, 10⁹ to 10¹² cfu, 10¹⁰ to 10¹² cfu, 10¹¹ to 10¹² cfu, or 10⁶ to 10¹⁰ cfu of bacteria or a bacterial strain described herein per mL.

Compositions described herein may comprise may at least about 0.01% by weight, at least about 0.05% by weight, at least about 0.1% by weight, at least about 0.2% by weight, at least about 0.3% by weight, at least about 0.4% by weight, at least about 0.5% by weight, at least about 0.6% by weight, at least about 0.7% by weight, at least about 0.8% by weight, at least about 0.9% by weight, at least about 1.0% by weight, at least about 1.5% by weight, at least about 2.0% by weight, at least about 3.0% by weight, at least about 4.0% by weight, at least about 5.0% by weight, at least about 6.0% by weight, at least about 7.0% by weight, at least about 8.0% by weight, at least about 9.0% by weight, at least about 10.0% by weight, at least about 11.0% by weight, at least about 12.0% by weight, at least about 13.0% by weight, at least about 14.0% by weight, at least about 15.0% by weight, at least about 16.0% by weight, at least about 17.0% by weight, at least about 18.0% by weight, at least about 19.0% by weight, at least about 20.0% by weight, at least about 25.0% by weight, at least about 30.0% by weight, at least about 35.0% by weight, at least about 40.0% by weight, at least about 45.0% by weight, or at least about 50.0% by weight of bacteria or bacterial strain described herein. In some embodiments, compositions can include from 0.01% to 30% by weight, from about 0.01% to 20% by weight, from 0.01% to 5% by weight, from 0.1% to 30% by weight, from 0.1% to 20% by weight, from 0.1% to about 15% by weight, from 0.1% to 10% by weight, from 0.1% to 5% by weight, from 0.2% to 5% by weight, from 0.3% to 5% by weight, from 0.4% to 5% by weight, from 0.5% to 5% by weight, or from 1% to 5% by weight of bacteria or bacterial strain described herein.

Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of a strain in Table 1 to another strain in Table 1 or a strain in Table 2 to another strain in Table 2. Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of a strain in Table 1 to a strain in Table 2. Compositions, including pharmaceutical compositions, described herein may comprise a ratio (cfu to cfu) of about: 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:200, 1:300, 1:400, 1:500, 1:600, 1:700, 1:800, 1:900 or about 1:1000 of multiple strains of Corynebacterium and/or Dolosigranulum pigrum.

### 3) Conditions

In some embodiments, provided herein are compositions for the prevention or treatment of a condition of the peripheral auditory system. In some embodiments, provided herein are compositions for the prevention or treatment of a condition of the middle ear. In some embodiments, provided herein are compositions for the prevention or treatment of otitis media (middle ear infection). In some embodiments, the otitis media is acute otitis media. Acute otitis media occurs suddenly, causes ear swelling and ear redness, traps fluid in the ear, and can result in fever and/or ear pain. In some embodiments, the otitis media is acute otitis media with effusion. Otitis media with effusion can provide mucus build up in the middle ear in a subject, and may negatively impact hearing (auditory perception) in a subject. In some embodiments, the otitis media is chronic otitis media with effusion. Chronic otitis media with effusion can provide mucus build up in the middle ear in a subject and may negatively impact hearing (auditory perception) in a subject. In some embodiments, compositions described comprise beneficial bacteria present in an amount sufficient for a reduction in incidence of colonization of a pathogenic bacteria. In some embodiments, compositions described comprise beneficial bacteria present in an amount sufficient for a reduction in incidence of colonization of Streptococcus pneumoniae, Haemophilus influenzae, Moraxella catarrhalis or any combination thereof. In some embodiments, a composition described herein is administered to a subject in an amount sufficient for reduction of ear swelling, ear redness, ear fluid, or ear mucus. In some embodiments, a composition described herein is administered to a subject in an amount sufficient for improvement in hearing (auditory perception). In some embodiments, the condition relates to a bacterial infection. Sources for bacterial infections for prevention or treatment with pharmaceutical compositions described herein include, without limitation, S. aureus (methicillin-resistant S. aureus (MRSA) and methicillin-sensitive S. aureus (MSSA)), S. pneumoniae, Pseudomonas aeruginosa, and Bordetella pertussis. Upper respiratory tract conditions for treatment or prevention following administration of composition described herein include, without limitation, allergic rhinitis or non-allergic rhinitis, including acute bacterial rhinosinusitis. Lower respiratory tract conditions for treatment or prevention following administration of composition described herein include, without limitation, asthma, tuberculosis, whooping cough (pertussis), pneumonia (including hospital-acquired pneumonia (HAP), ventilator-associated pneumonia (VAP), and community acquired pneumonia (CAP)), walking pneumonia, and bronchitis, lung cancer, cystic fibrosis, chronic obstructive pulmonary disease (COPD) (e.g., emphysema or chronic bronchitis), idiopathic pulmonary fibrosis (IPF), and interstitial lung disease (ILD). In some embodiments, a pharmaceutical composition described herein is administered to a S. aureus positive subject, optionally prior to, receiving a ventilator therapy. In further embodiments, the subject is diagnosed with COVID. In some embodiments, a pharmaceutical composition described herein is administered to a coronavirus (CoV) positive subject (e.g., SARS-COV-2, SARS-COV Tor2, and MERS-COV), optionally prior to, receiving a ventilator therapy. In some embodiments, the asthma is childhood asthma, adult onset asthma, occupational asthma, severe asthma, or seasonal asthma. In some embodiments, the lung cancer is small cell lung cancer or non-small cell lung cancer (NSCLC), including adenocarcinoma, squamous cell carcinoma, or large cell carcinoma. Exemplary lung cancers include, without limitation, primary pulmonary lymphoma, lymphangitic carcinomatosis, epithelioid hemangioendothelioma, or multiple cystic lung disease (MCLD), sarcomatoid carcinoma, adenosquamous carcinoma, salivary gland-type lung carcinoma, large cell neuroendocrine carcinoma, granular cell lung tumour, carcinoids, or atypical carcinoids. In some embodiments, the cancer is a lip cancer, tongue cancer, mouth cancer, oral cavity cancer, oropharyngeal cancer. Example cancers in this region include, without limitation, laryngeal cancer, nasopharyngeal cancer, oral squamous cell carcinoma, oropharyngeal squamous cell carcinoma, otic tumors, salivary gland tumors, and paranasal sinus cancer. In some embodiments, a pharmaceutical composition provided herein is an adjuvant to a treatment of respiratory conditions described herein. In some embodiments, cancer is stage I, II, III or IV. In some embodiments, the cancer is metastatic. In some embodiments, a pharmaceutical composition described herein is an adjuvant to chemotherapy for treatment of cancer. In some embodiments, the cancer is a solid cancer or hematopoietic cancer. Exemplary solid cancers include, without limitation, carcinoma and sarcoma. Exemplary hematopoietic cancers include, without limitation, leukemias, myelomas and lymphomas (including Hodgkin lymphomas, or non-Hodgkin lymphomas (NHLs)). Example chemotherapy agents include, without limitation, etoposide optionally with a platinum agent (cisplatin or carboplatin), 5-fluorouracil (5-FU), paclitaxel, docetaxel, hydroxyurea, methotrexate, bleomycin, and capecitabine. In some embodiments, at least one chemotherapy agent is used. In some embodiments, radiation (ionizing radiation) is administered. In some embodiments, radiation (ionizing radiation) and chemotherapy are administered. Therapies such as chemotherapy and radiation can induce an inflammatory response and compromise integrity of cell-cell junctions resulting in additional disease conditions, such as oral mucositis. In some embodiments, a pharmaceutical composition described herein is administered as an adjuvant with one or more checkpoint inhibitors for treatment of a cancer. Exemplary checkpoint inhibitors include, without limitation, anti-CTLA-4 antibody (e.g., ipilimumab), anti-PD-L1 antibody (e.g., atezolizumab), and anti-PD-1 antibody (e.g., nivolumab and pembrolizumab). In some embodiments, a pharmaceutical composition described herein is administered as treatment for a viral condition, or as an adjuvant to a therapy for treatment of a viral condition. In further embodiments, the virus is a virus of the respiratory tract. Exemplary virus of the respiratory tract include, without limitation, influenza A (e.g., HIN1 and HIN5), influenza B, an adenovirus, respiratory syncytial virus (RSV), enterovirus (EVs), human rhinovirus (HRV), human metapneumovirus (HMPV), human bocavirus (HBOV), coronavirus (CoV) (e.g., SARS-COV-2, SARS-COV Tor2, and MERS-COV), and parainfluenza virus (PIV). Exemplary therapies for viral conditions include, without limitation, oseltamivir, zanamivir, ribavirin, palivizumab, and aspirin. In some embodiments, a pharmaceutical composition described herein is administered to a SARS-COV-2 positive subject, optionally prior to, receiving a ventilator therapy. In some embodiments, a pharmaceutical composition used to treat or prevent a respiratory condition described herein comprises at least one species (or strain) listed in Table 1 or Table 2, or a mixture listed in Tables 5-8. In some embodiments, a pharmaceutical composition described herein is administered to a subject having a respiratory condition, optionally prior to, receiving a ventilator therapy. In some embodiments, a pharmaceutical composition used to treat or prevent a respiratory condition described herein comprises a strain of C. pseudodiphtheriticum and, optionally, a strain of D). pigrum. In some embodiments, a method for treating nasal colonization by at least one pathogenic microorganism in a subject is provided, the method comprising the steps of: administering a pharmaceutical composition to the subject, wherein the pharmaceutical composition comprises a Corynebacterium strain listed in Table 1, and a Dolosigranulum strain listed in Table 2. In some embodiments, a method for treating nasal colonization by at least one pathogenic microorganism in a subject is provided, the method comprising the steps of: administering a pharmaceutical composition to the subject, wherein the pharmaceutical composition comprises a C. pseudodiphtheriticum strain listed in Table 1, and a Dolosigranulum strain listed in Table 2, or a mixture listed in Tables 5-8. In some embodiments, provided herein are methods for reducing colonization of a subject's anterior nares or nasal cavity by a pathogenic microorganism, the method comprising the steps of: administering to a subject having a pathogenic microorganism in the subject's anterior nares or nasal cavity, a live, purified population of bacteria, wherein the live, purified population of bacteria comprises a plurality of strains of Corynebacterium pseudodiphtheriticum. Further provided herein are methods wherein the pathogenic microorganism comprises Staphylococcus aureus, Streptococcus pneumoniae, or Pseudomonas aeruginosa. Further provided herein are methods wherein the live, purified population of bacteria comprises a mixture listed in Tables 5-8. Further provided herein are methods wherein the live, purified population of bacteria comprises a mixture listed in Tables 5-8. Further provided herein are methods wherein the plurality of strains of C. pseudodiphtheriticum comprises JCM 1320 or ATCC 10700. Further provided herein are methods wherein the plurality of strains of C. pseudodiphtheriticum comprises JCM 1320 and ATCC 10700. Further provided herein are methods wherein the plurality of strains of C. pseudodiphtheriticum comprises strains selected from a strain listed in Table 1. Further provided herein are methods for reducing colonization of a subject's anterior nares or nasal cavity by a pathogenic microorganism, the method comprising the steps of: administering to a subject having a pathogenic microorganism in the subject's anterior nares or nasal cavity, a live, purified population of bacteria, wherein the live, purified population of bacteria comprises a plurality of species of Corynebacterium. Further provided herein are methods wherein the plurality of species of Corynebacterium comprises C. accolens, C. pseudodiphtheriticum, C. amycolatum, C. propinquum, C. glutamicum, or C. striatum. Further provided herein are methods wherein the pathogenic microorganism comprises Staphylococcus aureus, Streptococcus pneumoniae, or Pseudomonas aeruginosa. In some embodiments, provided herein are methods for restoration of a loss of smell, comprising: administering to a subject having an airway inflammatory condition, a live, purified population of bacteria, wherein the live, purified population of bacteria comprises a plurality of species of Corynebacterium.

### EXAMPLES

### Example 1: Bacterial Mixtures

i. Isolates and mixtures. Described herein are combinations of bacterial strains that can be used in generation of compositions, including pharmaceutical compositions for treatment of respiratory tract conditions. In the mixtures that follow, each of the live, purified strains are present in equal CFU amounts to other live, purified strains in each mixture.

First, mixtures having combinations of two strains are generated. Each mixture includes that two strains of Corynebacterium listed in Table 1, where an “X” denotes inclusion, are listed in the 44 mixtures (A1 to A44) in Table 5.

TABLE 5

| Mixture | KPL1989 | DSM44287 | 090104 | KPL1818 | DSM44278 | DSM6922 | DSM1567 | DSM44285 | DSM20300 | DSM20668 |
|---|---|---|---|---|---|---|---|---|---|---|
| A1 | X | X | | | | | | | | |
| A2 | X | | X | | | | | | | |
| A3 | X | | | X | | | | | | |
| A4 | X | | | | X | | | | | |
| A5 | X | | | | | X | | | | |
| A6 | X | | | | | | X | | | |
| A7 | X | | | | | | | X | | |
| A8 | X | | | | | | | | X | |
| A9 | X | | | | | | | | | X |
| A10 | | X | X | | | | | | | |
| A11 | | X | | X | | | | | | |
| A12 | | X | | | X | | | | | |
| A13 | | X | | | | X | | | | |
| A14 | | X | | | | | X | | | |
| A15 | | X | | | | | | X | | |
| A16 | | X | | | | | | | X | |
| A17 | | X | | | | | | | | X |
| A18 | | | X | X | | | | | | |
| A19 | | | X | | X | | | | | |
| A20 | | | X | | | X | | | | |
| A21 | | | X | | | | X | | | |
| A22 | | | X | | | | | X | | |
| A23 | | | X | | | | | | X | |
| A24 | | | X | | | | | | | X |
| A25 | | | | X | X | | | | | |
| A26 | | | | X | | X | | | | |
| A27 | | | | X | | | X | | | |
| A28 | | | | X | | | | X | | |
| A29 | | | | X | | | | | X | |
| A30 | | | | | X | X | | | | |
| A31 | | | | | X | | X | | | |
| A32 | | | | | X | | | X | | |
| A33 | | | | | X | | | | X | |
| A34 | | | | | X | | | | | X |
| A35 | | | | | | X | X | | | |
| A36 | | | | | | X | | X | | |
| A37 | | | | | | X | | | X | |
| A38 | | | | | | X | | | | X |
| A39 | | | | | | | X | X | | |
| A40 | | | | | | | | | X | |
| A41 | | | | | | | | | | X |
| A42 | | | | | | | | X | X | |
| A43 | | | | | | | | X | | X |
| A44 | | | | | | | | | X | X |

Second, mixtures are made that includes two strains of D. pigrum listed in Table 2, where an “X” denotes inclusion, are listed in the 66 mixtures (B1 to B66) in Table 6.

TABLE 6

| | | CDC39- | CDC2949- | CDC4294- | CDC4420- | CDC4545- | CDC4709- | CDC4199- | CDC4791- | CDC4791- | | |
|---|---|---|---|---|---|---|---|---|---|---|---|---|
| Mixture | KLP1914 | 95 | 98 | 98 | 98 | 98 | 98 | 99 | 99 | 99 | AMBR11 | AMBR12 |
| B1 | X | X | | | | | | | | | | |
| B2 | X | | X | | | | | | | | | |
| B3 | X | | | X | | | | | | | | |
| B4 | X | | | | X | | | | | | | |
| B5 | X | | | | | X | | | | | | |
| B6 | X | | | | | | X | | | | | |
| B7 | X | | | | | | | X | | | | |
| B8 | X | | | | | | | | X | | | |
| B9 | X | | | | | | | | | X | | |
| B10 | X | | | | | | | | | | X | |
| B11 | X | | | | | | | | | | | X |
| B12 | | X | X | | | | | | | | | |
| B13 | | X | | X | | | | | | | | |
| B14 | | X | | | X | | | | | | | |
| B15 | | X | | | | X | | | | | | |
| B16 | | X | | | | | X | | | | | |
| B17 | | X | | | | | | X | | | | |
| B18 | | X | | | | | | | X | | | |
| B19 | | X | | | | | | | | X | | |
| B20 | | X | | | | | | | | | X | |
| B21 | | X | | | | | | | | | | X |
| B22 | | | X | X | | | | | | | | |
| B23 | | | X | | X | | | | | | | |
| B24 | | | X | | | X | | | | | | |
| B25 | | | X | | | | X | | | | | |
| B26 | | | X | | | | | X | | | | |
| B27 | | | X | | | | | | X | | | |
| B28 | | | X | | | | | | | X | | |
| B29 | | | X | | | | | | | | X | |
| B30 | | | X | | | | | | | | | X |
| B31 | | | | X | X | | | | | | | |
| B32 | | | | X | | X | | | | | | |
| B33 | | | | X | | | X | | | | | |
| B34 | | | | X | | | | X | | | | |
| B35 | | | | X | | | | | X | | | |
| B36 | | | | X | | | | | | X | | |
| B37 | | | | X | | | | | | | X | |
| B38 | | | | X | | | | | | | | X |
| B39 | | | | | X | X | | | | | | |
| B40 | | | | | X | | X | | | | | |
| B41 | | | | | X | | | X | | | | |
| B42 | | | | | X | | | | X | | | |
| B43 | | | | | X | | | | | X | | |
| B44 | | | | | X | | | | | | X | |
| B45 | | | | | X | | | | | | | X |
| B46 | | | | | | X | X | | | | | |
| B47 | | | | | | X | | X | | | | |
| B48 | | | | | | X | | | X | | | |
| B49 | | | | | | X | | | | X | | |
| B50 | | | | | | X | | | | | X | |
| B51 | | | | | | X | | | | | | X |
| B52 | | | | | | | X | X | | | | |
| B53 | | | | | | | X | | X | | | |
| B54 | | | | | | | X | | | X | | |
| B55 | | | | | | | X | | | | X | |
| B56 | | | | | | | X | | | | | X |
| B57 | | | | | | | | X | X | | | |
| B58 | | | | | | | | X | | X | | |
| B59 | | | | | | | | X | | | X | |
| B60 | | | | | | | | X | | | | X |
| B61 | | | | | | | | | X | X | | |
| B62 | | | | | | | | | X | | X | |
| B63 | | | | | | | | | X | | | X |
| B64 | | | | | | | | | | X | X | |
| B65 | | | | | | | | | | X | | X |
| B66 | | | | | | | | | | | X | X |

Third, mixtures of Corynebacterium listed in Table 1 are each combined with one of the 12 strains of D. pigrum listed in Table 2, where an “X” denotes inclusion, are listed in the 66 mixtures (C1 to C130) in Table 7.

TABLE 7

| | Corynebacterium | | | | | | |
|---|---|---|---|---|---|---|---|
| | strain | | CDC39- | CDC2949- | CDC4294- | CDC4420- | CDC4545- |
| Mixture | (below) | KLP1914 | 95 | 98 | 98 | 98 | 98 |
| C1 | KPL1989 | X | | | | | |
| C2 | DSM44287 | X | | | | | |
| C3 | 090104 | X | | | | | |
| C4 | KPL1818 | X | | | | | |
| C5 | DSM44278 | X | | | | | |
| C6 | DSM6922 | X | | | | | |
| C7 | DSM1567 | X | | | | | |
| C8 | DSM44285 | X | | | | | |
| C9 | DSM20300 | X | | | | | |
| C10 | DSM20668 | X | | | | | |
| C11 | KPL1989 | | X | | | | |
| C12 | DSM44287 | | X | | | | |
| C13 | 090104 | | X | | | | |
| C14 | KPL1818 | | X | | | | |
| C15 | DSM44278 | | X | | | | |
| C16 | DSM6922 | | X | | | | |
| C17 | DSM1567 | | X | | | | |
| C18 | DSM44285 | | X | | | | |
| C19 | DSM20300 | | X | | | | |
| C20 | DSM20668 | | X | | | | |
| C21 | KPL1989 | | | X | | | |
| C22 | DSM44287 | | | X | | | |
| C23 | 090104 | | | X | | | |
| C24 | KPL1818 | | | X | | | |
| C25 | DSM44278 | | | X | | | |
| C26 | DSM6922 | | | X | | | |
| C27 | DSM1567 | | | X | | | |
| C28 | DSM44285 | | | X | | | |
| C29 | DSM20300 | | | X | | | |
| C30 | DSM20668 | | | X | | | |
| C41 | KPL1989 | | | | X | | |
| C42 | DSM44287 | | | | X | | |
| C43 | 090104 | | | | X | | |
| C44 | KPL1818 | | | | X | | |
| C45 | DSM44278 | | | | X | | |
| C46 | DSM6922 | | | | X | | |
| C47 | DSM1567 | | | | X | | |
| C48 | DSM44285 | | | | X | | |
| C49 | DSM20300 | | | | X | | |
| C50 | DSM20668 | | | | X | | |
| C51 | KPL1989 | | | | | X | |
| C52 | DSM44287 | | | | | X | |
| C53 | 090104 | | | | | X | |
| C54 | KPL1818 | | | | | X | |
| C55 | DSM44278 | | | | | X | |
| C56 | DSM6922 | | | | | X | |
| C57 | DSM1567 | | | | | X | |
| C58 | DSM44285 | | | | | X | |
| C59 | DSM20300 | | | | | X | |
| C60 | DSM20668 | | | | | X | |
| C61 | KPL1989 | | | | | | X |
| C62 | DSM44287 | | | | | | X |
| C63 | 090104 | | | | | | X |
| C64 | KPL1818 | | | | | | X |
| C65 | DSM44278 | | | | | | X |
| C66 | DSM6922 | | | | | | X |
| C67 | DSM1567 | | | | | | X |
| C68 | DSM44285 | | | | | | X |
| C69 | DSM20300 | | | | | | X |
| C70 | DSM20668 | | | | | | X |
| C71 | KPL1989 | | | | | | |
| C72 | DSM44287 | | | | | | |
| C73 | 090104 | | | | | | |
| C74 | KPL1818 | | | | | | |
| C75 | DSM44278 | | | | | | |
| C76 | DSM6922 | | | | | | |
| C77 | DSM1567 | | | | | | |
| C78 | DSM44285 | | | | | | |
| C79 | DSM20300 | | | | | | |
| C80 | DSM20668 | | | | | | |
| C81 | KPL1989 | | | | | | |
| C82 | DSM44287 | | | | | | |
| C83 | 090104 | | | | | | |
| C84 | KPL1818 | | | | | | |
| C85 | DSM44278 | | | | | | |
| C86 | DSM6922 | | | | | | |
| C87 | DSM1567 | | | | | | |
| C88 | DSM44285 | | | | | | |
| C89 | DSM20300 | | | | | | |
| C90 | DSM20668 | | | | | | |
| C91 | KPL1989 | | | | | | |
| C92 | DSM44287 | | | | | | |
| C93 | 090104 | | | | | | |
| C94 | KPL1818 | | | | | | |
| C95 | DSM44278 | | | | | | |
| C96 | DSM6922 | | | | | | |
| C97 | DSM1567 | | | | | | |
| C98 | DSM44285 | | | | | | |
| C99 | DSM20300 | | | | | | |
| C100 | DSM20668 | | | | | | |
| C101 | KPL1989 | | | | | | |
| C102 | DSM44287 | | | | | | |
| C103 | 090104 | | | | | | |
| C104 | KPL1818 | | | | | | |
| C105 | DSM44278 | | | | | | |
| C106 | DSM6922 | | | | | | |
| C107 | DSM1567 | | | | | | |
| C108 | DSM44285 | | | | | | |
| C109 | DSM20300 | | | | | | |
| C110 | DSM20668 | | | | | | |
| C111 | KPL1989 | | | | | | |
| C112 | DSM44287 | | | | | | |
| C113 | 090104 | | | | | | |
| C114 | KPL1818 | | | | | | |
| C115 | DSM44278 | | | | | | |
| C116 | DSM6922 | | | | | | |
| C117 | DSM1567 | | | | | | |
| C118 | DSM44285 | | | | | | |
| C119 | DSM20300 | | | | | | |
| C120 | DSM20668 | | | | | | |
| C121 | KPL1989 | | | | | | |
| C122 | DSM44287 | | | | | | |
| C123 | 090104 | | | | | | |
| C124 | KPL1818 | | | | | | |
| C125 | DSM44278 | | | | | | |
| C126 | DSM6922 | | | | | | |
| C127 | DSM1567 | | | | | | |
| C128 | DSM44285 | | | | | | |
| C129 | DSM20300 | | | | | | |
| C130 | DSM20668 | | | | | | |

| | CDC4709- | CDC4199- | CDC4791- | CDC4791- | | |
|---|---|---|---|---|---|---|
| Mixture | 98 | 99 | 99 | 99 | AMBR11 | AMBR12 |
| C1 | | | | | | |
| C2 | | | | | | |
| C3 | | | | | | |
| C4 | | | | | | |
| C5 | | | | | | |
| C6 | | | | | | |
| C7 | | | | | | |
| C8 | | | | | | |
| C9 | | | | | | |
| C10 | | | | | | |
| C11 | | | | | | |
| C12 | | | | | | |
| C13 | | | | | | |
| C14 | | | | | | |
| C15 | | | | | | |
| C16 | | | | | | |
| C17 | | | | | | |
| C18 | | | | | | |
| C19 | | | | | | |
| C20 | | | | | | |
| C21 | | | | | | |
| C22 | | | | | | |
| C23 | | | | | | |
| C24 | | | | | | |
| C25 | | | | | | |
| C26 | | | | | | |
| C27 | | | | | | |
| C28 | | | | | | |
| C29 | | | | | | |
| C30 | | | | | | |
| C41 | | | | | | |
| C42 | | | | | | |
| C43 | | | | | | |
| C44 | | | | | | |
| C45 | | | | | | |
| C46 | | | | | | |
| C47 | | | | | | |
| C48 | | | | | | |
| C49 | | | | | | |
| C50 | | | | | | |
| C51 | | | | | | |
| C52 | | | | | | |
| C53 | | | | | | |
| C54 | | | | | | |
| C55 | | | | | | |
| C56 | | | | | | |
| C57 | | | | | | |
| C58 | | | | | | |
| C59 | | | | | | |
| C60 | | | | | | |
| C61 | | | | | | |
| C62 | | | | | | |
| C63 | | | | | | |
| C64 | | | | | | |
| C65 | | | | | | |
| C66 | | | | | | |
| C67 | | | | | | |
| C68 | | | | | | |
| C69 | | | | | | |
| C70 | | | | | | |
| C71 | X | | | | | |
| C72 | X | | | | | |
| C73 | X | | | | | |
| C74 | X | | | | | |
| C75 | X | | | | | |
| C76 | X | | | | | |
| C77 | X | | | | | |
| C78 | X | | | | | |
| C79 | X | | | | | |
| C80 | X | | | | | |
| C81 | | X | | | | |
| C82 | | X | | | | |
| C83 | | X | | | | |
| C84 | | X | | | | |
| C85 | | X | | | | |
| C86 | | X | | | | |
| C87 | | X | | | | |
| C88 | | X | | | | |
| C89 | | X | | | | |
| C90 | | X | | | | |
| C91 | | | X | | | |
| C92 | | | X | | | |
| C93 | | | X | | | |
| C94 | | | X | | | |
| C95 | | | X | | | |
| C96 | | | X | | | |
| C97 | | | X | | | |
| C98 | | | X | | | |
| C99 | | | X | | | |
| C100 | | | X | | | |
| C101 | | | | X | | |
| C102 | | | | X | | |
| C103 | | | | X | | |
| C104 | | | | X | | |
| C105 | | | | X | | |
| C106 | | | | X | | |
| C107 | | | | X | | |
| C108 | | | | X | | |
| C109 | | | | X | | |
| C110 | | | | X | | |
| C111 | | | | | X | |
| C112 | | | | | X | |
| C113 | | | | | X | |
| C114 | | | | | X | |
| C115 | | | | | X | |
| C116 | | | | | X | |
| C117 | | | | | X | |
| C118 | | | | | X | |
| C119 | | | | | X | |
| C120 | | | | | X | |
| C121 | | | | | | X |
| C122 | | | | | | X |
| C123 | | | | | | X |
| C124 | | | | | | X |
| C125 | | | | | | X |
| C126 | | | | | | X |
| C127 | | | | | | X |
| C128 | | | | | | X |
| C129 | | | | | | X |
| C130 | | | | | | X |

Fourth, a mixture of multiple Corynebacterium strains from Table 1 is made. In this example, 2, 3, 4, or 5 strains are from difference species of Corynebacterium are in the composition. Strains are selected from: C. pseudodiphtheriticum ATCC 10700 and/or JCM 1320; C. amycolatum ATCC 49358; C. glutamicum ATCC 13032; and C. striatum ATCC 6940. The compositions may include ATCC 10700 and/or JCM 1320 in addition to the strains from species other than ATCC 10700 and/or JCM 1320.

Fifth, a mixture of two Corynebacterium pseudodiphtheriticum strains from Table 1 is made: ATCC 10700 and JCM 1320, “mixture D1.”

Sixth, mixture D1 is combined with one of the 12 strains of D. pigrum listed in Table 2, where an “X” denotes inclusion, are listed in the 66 mixtures (E1 to E12) in Table 8.

TABLE 8

| | Mixture | | | | | | |
|---|---|---|---|---|---|---|---|
| | D1 | | | | | | |
| | (ATCC10700 | | CDC39- | CDC2949- | CDC4294- | CDC4420- | CDC4545- |
| Mixture | and JCM1320) | KLP1914 | 95 | 98 | 98 | 98 | 98 |
| E1 | X | X | | | | | |
| E2 | X | | X | | | | |
| E3 | X | | | X | | | |
| E4 | X | | | | X | | |
| E5 | X | | | | | X | |
| E6 | X | | | | | | X |
| E7 | X | | | | | | |
| E8 | X | | | | | | |
| E9 | X | | | | | | |
| E10 | X | | | | | | |
| E11 | X | | | | | | |
| E12 | X | | | | | | |

| | CDC4709- | CDC4199- | CDC4791- | CDC4791- | | |
|---|---|---|---|---|---|---|
| Mixture | 98 | 99 | 99 | 99 | AMBR11 | AMBR12 |
| E1 | | | | | | |
| E2 | | | | | | |
| E3 | | | | | | |
| E4 | | | | | | |
| E5 | | | | | | |
| E6 | | | | | | |
| E7 | X | | | | | |
| E8 | | X | | | | |
| E9 | | | X | | | |
| E10 | | | | X | | |
| E11 | | | | | X | |
| E12 | | | | | | X |

Seventh mixtures of Corynebacterium listed in Table 1 are combined to include at least one strain of each of C. pseudodiphtheriticum, C. accolens, and C. amycolatum. For example: ATCC 10700 and/or JCM 1320 plus ATCC 49726 and ATCC 49368.

iii. Cultivation from frozen stocks. Bacterial strains are grown at 37° C. with 5% CO2. D. pigrum strains are cultivated from frozen stocks on BBL Columbia Colistin-Nalidixic Acid (CNA) agar with 5% sheep blood (BD Diagnostics) for 2 days. Corynebacterium species are cultivated from frozen stocks on BHI agar (e.g., for C. pseudodiphtheriticum and C. propinquum) or BHI agar supplemented with 1% Tween 80 (e.g., for C. accolens) for 1 day. Resuspensions described below are made by harvesting colonies from agar medium and resuspending in 1× phosphate buffered saline (PBS).

### Example 2: MRSA Model Systems

i. Contact-Dependent Assays. Clinical isolates of C. pseudodiphtheriticum and D. pigrum taken from subjects (e.g., from strains listed in Table 1 and Table 2, and mixtures from Example 1) are assayed to determine if anti-S. aureus activity is dependent on direct physical contact between the bacteria. Briefly, a sterile 0.2 μm filter disk is placed on top of the BHIT agar (Brain Heart Infusion (BHI) agar (Becton Dickinson)) seeded with one of the S. aureus strains provided in Table 4 (JE2, LAC, Mu50, or USA 900). Each clinical isolate in a suspension is individually spotted on top of the filter disk so that none of the cell suspension physically touched the S. aureus seeded agar plate. Plates are incubated at 28° C. and visually assessed at 24, 72, and 120 hours for the absence or presence of a zone of clearance (ZOC). The absence of a ZOC in the presence of a filter disk indicates that physical contact is necessary for anti-S. aureus activity against the corresponding most sensitive S. aureus strain.

ii. Conditioned Cell Free Medium (CCFM) Preparation and Disk Diffusion Assays. Clinical isolates that produce contact-independent bactericidal anti-S. aureus activity (e.g., from strains listed in Table 1, Table 2, and mixtures from Example 1) are independently cultured in 10 mL BHIT broth overnight at 37° ° C. with shaking at 190 rpm. Cultures are pelleted by centrifugation, and the supernatant is filter-sterilized with a 2 μm filter (Corning). One-milliliter of sterile supernatant is retained, and the remaining supernatant is concentrated (50×) with ammonium sulfate precipitation. For heat-treatments, 50 μL aliquots of unconcentrated or 50×CCFM are incubated at 90ºC for 10 minutes, then allowed to cool. For the disk diffusion assays, the S. aureus strain that is most sensitive to the corresponding inhibitory activity is cultured on BHI agar overnight at 37° C. The following day, the plate-grown cells are recovered and diluted to 1×10\hat{ }8 cells/ml (OD600 of 0.1) in BHI broth. A sterile swab is then used to spread the S. aureus cell suspension on BHIT agar as a lawn. The plate is allowed to dry in a laminar flow hood for 30 minutes. Next, a sterile 5 mm diffusion disk is placed on top of the S. aureus lawn, and 50 μL of unconcentrated CCFM or 50×CCFM is inoculated onto the disk. Plates are incubated at 28° C., and images are taken after 72 hours of incubation.

iii. S. aureus Infection and CCFM Treatment of Galleria mellonella Caterpillars.

Staphylococcus aureus strains JE2, LAC, Mu50, or USA 900 are cultured overnight on BHI agar at 37° C. The following day, S. aureus cells are recovered and diluted to 1×10\hat{ }8 cells/ml (OD600=0.1) in PBS. Total CFU are then further adjusted to obtain the required doses; i.e., 10\hat{ }7 CFU or 10\hat{ }6 CFU in 5 L of PBS+0.01% bromophenol dye. For infections, Galleria mellonella caterpillars (Vanderhorst Wholesale Inc) are utilized within 1 day of receipt. Caterpillars between 200 and 300 mg are chosen for infection. Briefly, 5 μL of inoculum that contained 10\hat{ }7 or 10\hat{ }6 total CFU of S. aureus are injected into the last left proleg using a 10 μL glass syringe (Hamilton) fitted with a 31G needle. For caterpillars that are treated with CCFM obtains from strains in Table 1, Table 2, and mixtures from Example 1, the caterpillars are maintained at room temperature for 1 hour following the S. aureus injection, then refrigerated at 4° C. for 12 minutes and then injected with 5 μL of freshly prepared 50×CCFM from the clinical isolate (treated) or 50× concentrated BHIT (sham treated). These injections are into the last right proleg. All caterpillars are incubated at 37ºC, and survival was monitored over 120 hours. Untouched, and PBS injected caterpillars are included as controls.

iv. Intranasal colonization assay for Methicillin-Resistant Staphylococcus aureus in Mice. Staphylococcus aureus strains JE2, LAC, Mu50, or USA 900 are cultured at 37° C. in either Todd-Hewitt broth (THB) or on Todd-Hewitt agar (THA) (Difco). Brain Heart Infusion (BHI) (Difco) broth and agar are used to grow C. pseudodiphtheriticum and D. pigrum strains (e.g., from strains listed in Table 1, Table 2, and Example 1). CDI mice (Charles River Laboratories, Wilmington, MA) are obtained. Mice are inoculated intranasally with 10 μl droplet of the inocula at the indicated concentrations. Mice are administered 1×10\hat{ }9 CFU total of the bacteria. CDI mice are inoculated intranasally with: (i) C. pseudodiphtheriticum, (ii) D. pigrum, (iii) C. pseudodiphtheriticum and D). pigrum, or (iv) PBS. After two days, the mice are administered streptomycin-resistant MRSA (JE2, LAC, Mu50, or USA 900) by the intranasal route, and sacrificed after another 2 days. For bacterial enumeration, the mice are euthanized using isoflurane followed by cervical dislocation, and the nasal tissue is homogenized and vortexed for 5 min in PBS, and the homogenate is plated on THA with or without streptomycin after appropriate serial dilutions. Bacterial identification is based on antibiotic resistance patterns, colony morphology, and color.

### Example 3: Pneumonia Model Systems

i. Growth assay of S. pneumoniae in cell-free conditioned liquid medium. After growth in BHI, D. pigrum or C. pseudodiphtheriticum cells (e.g., from strains listed in Table 1, Table 2, and mixtures from Example 1) are removed with a 0.22-μM sterile filter yielding cell-free conditioned medium (CFCM). The pH of the CFCM is adjusted using 2N H2SO4 and 10M KOH to match that of BHI broth alone within 0.02 pH units. S. pneumoniae strains TIGR4, DBL5, and M270-8 (see Table 4) are each grown on BBL Columbia CNA agar with 5% sheep blood for 1 day, harvested with a sterile cotton swab, resuspended to an OD600 of 0.30 in 1×PBS, inoculated at 1:100 into both of D. pigrum or C. pseudodiphtheriticum CFCM and BHI broth and grown for 19-20 hour at 37° C. in static (S. pneumoniae) culture under atmospheric conditions. Growth yield is quantified as OD600 absorbance.

ii. Growth assay for S. pneumoniae in conditioned by mono- vs. co-culture medium. D. pigrum and C. pseudodiphtheriticum strains (e.g., from strains listed in Table 1, Table 2, and mixtures from Example 1) are grown from freezer stocks. Cells are harvested with sterile cotton swabs and resuspended in sterile PBS to an OD600 nm of 0.5. Cells are then spotted in 100 μl of 1:1 mixed resuspension on a polycarbonate membrane on BHI agar medium containing 400 U/mL bovine liver catalase. After 2 days of growth, the polycarbonate membrane with D. pigrum and/or C. pseudodiphtheriticum is removed from each plate leaving cell-free conditioned agar medium. S. pneumoniae is grown overnight on BBL Columbia CNA agar with 5% sheep blood using a sterile cotton swab, a lawn is streaked onto the cell-free conditioned agar medium and allowed to grow for 24 hours. Growth/inhibition is assessed daily and photographically recorded.

iii. Mouse model. 6- to 8-week-old FVB/N mice are orally gavaged with 200 μL of either (i) C. pseudodiphtheriticum, (ii) D. pigrum, (iii) C. pseudodiphtheriticum and D). pigrum (e.g., from strains listed in Table 1, Table 2, and mixtures from Example 1), or (iv) sterile water (vehicle) (for the bacteria: 1×10\hat{ }9 colony-forming units (CFUs)/mL) immediately before procedure. Pneumonia is induced via direct intratracheal instillation of Pseudomonas aeruginosa (ATCC 27853) or S. pneumoniae (TIGR4, M270-8, or DBL5). Under isoflurane anesthesia, mice receive a midline cervical incision, and P. aeruginosa or S. pneumoniae is introduced into the trachea via a 29-gauge syringe. Forty microliters of 4×10\hat{ }8 CFUs of bacteria diluted in sterile saline is used. Mice are then held vertically for 5 seconds to enhance the delivery into the lungs. Sham mice are treated identically except that they receive an intratracheal injection of saline. All mice receive antibiotic therapy (gentamicin 0.2 mg/mL, subcutaneously) after the surgery to mimic clinical setting. Animals are killed at either 12 or 24 hours (for acute studies) or followed 7 days for survival. For acute studies, mice receive a single dose of (i) C. pseudodiphtheriticum, (ii) D. pigrum, or (iii) C. pseudodiphtheriticum and D. pigrum. For survival studies, mice are treated with (i) C. pseudodiphtheriticum, (ii) D. pigrum, or (iii) C. pseudodiphtheriticum and D). pigrum daily for 7 days and receive antibiotic treatment at 0, 12, and 24 hours. Lungs are tested for the presence of P. aeruginosa or S. pneumoniae.

### Example 4: Rhinitis Model System

Female BALB/cA mice 2 months old are used. Animals are fed a standard rodent chow diet in a temperature-controlled room (23 degrees C.) on a 12 hour light/dark cycle. The immune response of the mice is suppressed by subcutaneous injections of hydrocortisone (Hydrocortisone hemisuccinate 100, Polfa, PL, 100 mg/kg/day) at day 0 and day 4. S. aureus (JE2, LAC, Mu50, or USA 900) is applied intranasally on day 5. Bacterial treatment is performed on day 10. Forty microliters of 4×10\hat{ }8 CFUs of bacteria diluted in sterile saline is used. Mice receive a single dose of (i) C. pseudodiphtheriticum, (ii) D. pigrum, or (iii) (′. pseudodiphtheriticum and D. pigrum (e.g., from strains listed in Table 1, Table 2, and mixtures from Example 1); or (iv) saline (vehicle). Animals are killed at either 12 or 24 hours or followed 7 days for survival, and tissue samples are taken. Blood and internal organ (e.g., lungs, kidney, liver and spleen) samples are tested for the presence of S. aureus; nasal epithelium is tested for carriage of S. aureus in control and experimental animals.

### Example 5: Corynebacterium Growth Assay

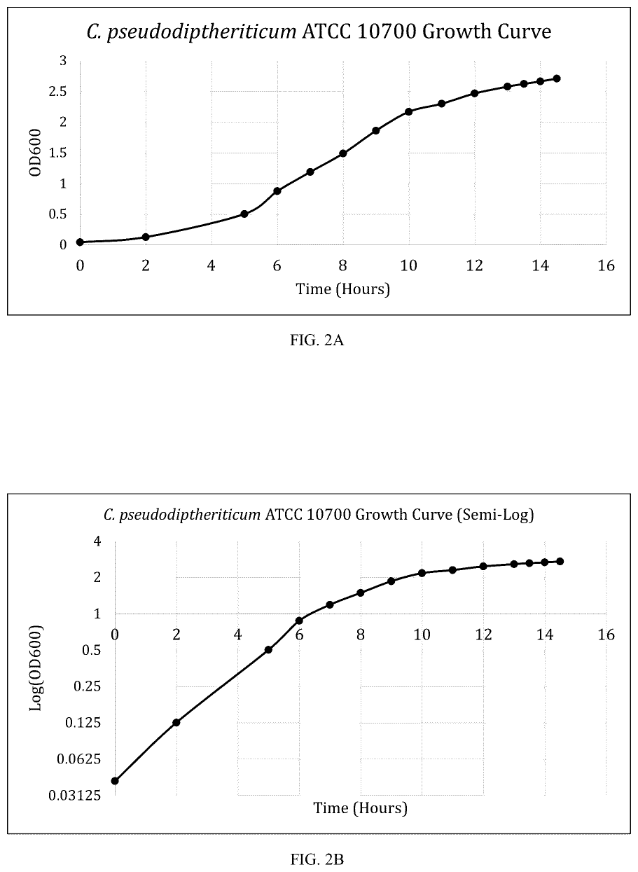

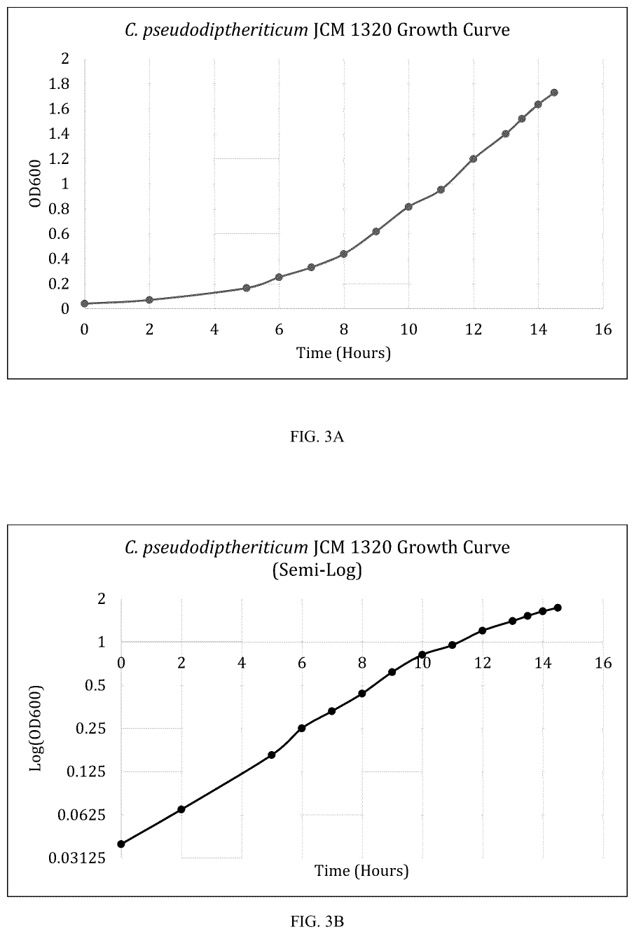

The following was performed to assay growth rate attributes for C. pseudodiptheriticum strains. 9 mL of Columbia 1% Tween broth was inoculated with C. pseudodiptheriticum colonies and incubated overnight at 37 degrees Celsius, shaking at 200 rpm. From the overnight culture, an OD600 measurement was taken and 30 mL of fresh Columbia 1% Tween broth was inoculated to reach a starting OD600 of 0.04, cultures were incubated at 37 degrees Celsius shaking at 200 rpm. OD600 measurements were taken over a 14.5 hour time period. Results of OD600 measurements for growth of ATCC 10700 are show in in FIG. 2A, and shown as Log(OD600) for the Y axis in FIG. 2B. As can be seen, the doubling time during log phase (hours 2 to 6) was about 86 minutes. Results of OD600 measurements for growth of JCM 1320 are show in in FIG. 3A, and shown as Log(OD600) for the Y axis in FIG. 3B. As can be seen, the doubling time during log phase (hours 2 to 6) was about 129 minutes.

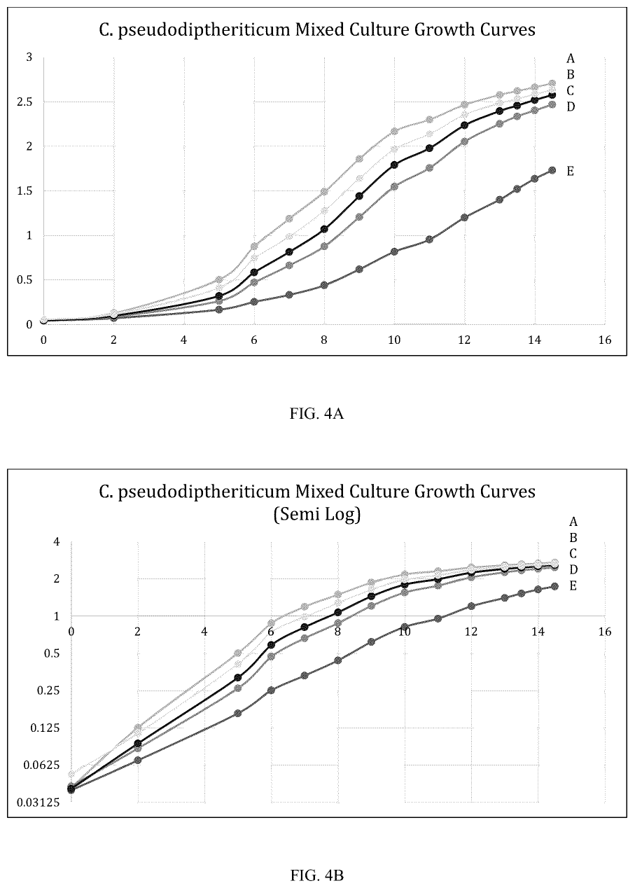

To assay co-culture growth, starting inoculum was varied to achieve mixed cultures while maintaining starting OD600 of 0.04. For example, diluting overnight culture to 0.04 for 400 μL for a 75% JCM 1320 25% ATCC 10700 mix, required 300 μL from the JCM 1320 culture and 100 μL from the ATCC 10700 culture. OD600 measurements were taken over a 14.5 hour time period. Results of OD600 measurements for growth are shown in in FIG. 4A, where samples A, B, C, D, and E are 100% ATCC 10700, 25% JCM 1320 and 75% ATCC 10700, 50% JCM 1320 and 50% ATCC 10700, 75% JCM 1320 and 25% ATCC 10700, and 100% JCM 1320, respectively. Results of Log(OD600) measurements for growth are shown in in FIG. 4B, with the same sample order ordering of data trendlines as in FIG. 4A. Doubling times were as follows: 100% ATCC 10700 [86 minutes], 25% JCM 1320 and 75% ATCC 10700 [89 minutes], 50% JCM 1320 and 50% ATCC 10700 [91 minutes], 75% JCM 1320 and 25% ATCC 10700 [98 minutes], and 100% JCM 1320 [129 minutes]. Doubling time appeared primarily driven by the ATCC 10700 strain. Final stationary phase cell density of mixed cultures was within similar range (OD₆₀₀±0.1).

### Example 6: Consortia Growth Assays

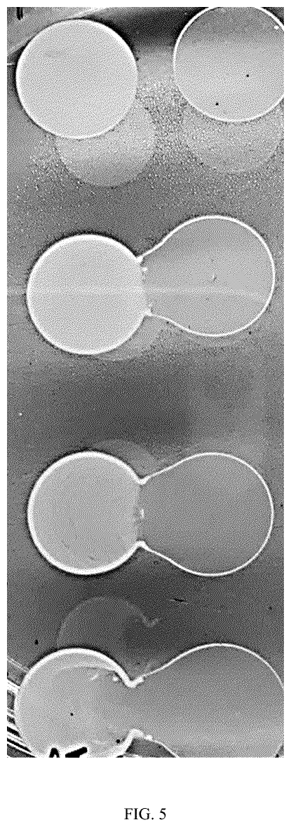

The following was performed to assay co-culture attributes for strains of C. pseudodiptheriticum. First, a direct co-culture of C. pseudodiptheriticum strains ATCC 10700 and JCM 1320 was performed. Both C. pseudodiptheriticum strains were spotted in close proximity at the same time on a Columbia 1% Tween agar plate: four spottings of ATCC 10700 on the left, and for spottings of JCM 1320 on the right. An image capture was taken after 24 hours from spotting, as shown in FIG. 5. As can be seen in FIG. 5, the cultures do not display features of antagonism between the different strains. This is an unexpected result, as co-culturing of C. pseudodiptheriticum clinical isolates has not been reported to date.

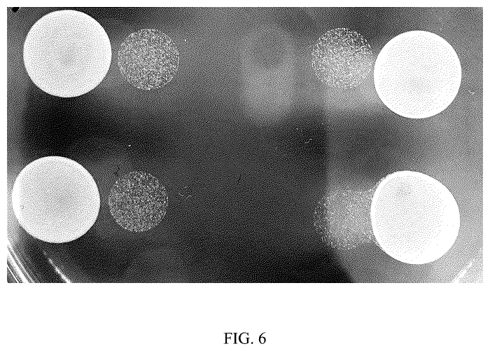

Second, a direct co-culture of C. pseudodiptheriticum strain ATCC 10700 or JCM 1320, and D. pigrum was performed. An image capture was taken after 24 hours from spotting, as shown in FIG. 6. Referring to FIG. 6, on the left is a column after two spottings of ATCC 10700 and just to the right of that is a column after two spottings of D. pigrum. On the right of the image is a column after two spottings of D. pigrum and just to the right of that is a column after two spottings of JCM 1320. As can be seen in FIG. 6, the cultures do not display features of antagonism between the different strains.

### Example 7: Hearing Screening Assay

i. Screening children ages 3 through 10 years. Set selector switch to “Right” and present 40 dB at 1000 Hz. Turn dial to 20 dB and present tones at 1000, 2000, and 4000 Hz. Turn selector switch to “Left” and present tones at 4000, 2000, and 1000 Hz. Set dial to 25 dB and present tone at 500 Hz; next, turn selector switch to “Right” and present tone at 500 Hz. Present tones for one to two seconds; you may present the tone twice consecutively if needed for each screening frequency. If the child did not hear one or more tones in either ear, perform an immediate rescreen by repeating the entire pure tone series, preferably with a different screener and audiometer. Document screening results.

ii. Screening subjects ages 11 through 20 years and over. Set selector switch to “Right” and present 40 dB at 1000 Hz. Turn dial to 20 dB and present tones at 1000, 2000, 4000, and 6000 Hz. Turn selector switch to “Left” and present tones at 6000, 4000, 2000, and 1000 Hz. Set dial to 25 dB and present tone at 500 Hz; next, turn selector switch to “Right” and present tone at 500 Hz. Present tones for one to two seconds; you may present the tone twice consecutively if needed for each screening frequency. If the subject did not hear one or more tones in either ear, perform an immediate rescreen by repeating the entire pure tone series, preferably with a different screener and audiometer. Document screening results.

While some embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treatment of a middle ear infection, comprising:
administering to a subject a live, purified population of bacteria, comprising:
a plurality of strains of Dolosigramilum pigrum, wherein the live, purified population of bacteria is present in an amount sufficient for treatment of a middle ear infection.

2. The method of claim 1, wherein the middle ear infection is otitis media.

3. The method of claim 2, wherein the otitis media is acute otitis media, otitis media with effusion, or chronic otitis media with effusion.

4. The method of claim 1, wherein the live, purified population of bacteria is further present in an amount sufficient for reduction of ear pain.

5. The method of claim 1, wherein the live, purified population of bacteria is further present in an amount sufficient for reduction of fever.

6. The method of claim 1, wherein the live, purified population of bacteria is present in an amount sufficient for reduction in colonization of a pathogenic bacteria in a nasal cavity of the subject.

7. The method of claim 1, wherein the live, purified population of bacteria is present in an amount sufficient for a reduction in colonization of Streptococcus pneumoniae, Haemophilus influenzae, or Moraxella catarrhalis in the middle ear.

8. The method of claim 1, wherein the plurality of strains of Dolosigramilum pigrum comprises LMG P-31124 or LMG P-31154.

9. The method of claim 1, wherein the live, purified population of bacteria further comprises at least one strain of Corynebacterium.

10. The method of claim 9, wherein the at least one strain of Corynebacterium comprises at least one strain of Corynebacterium pseudodiphtheriticum.

11. The method of claim 10, wherein the at least one strain of Corynebacterium pseudodiphtheriticum comprises JCM 1320 or ATCC 10700.

12. The method of claim 10, wherein the at least one strain of Corynebacterium pseudodiphtheriticum comprises JCM 1320 and ATCC 10700.

13. The method of claim 1, wherein the live, purified population of bacteria is administered intranasally.

14. The method of claim 13, wherein the live, purified population of bacteria is administered intranasally by spraying.

15. The method of claim 1, wherein the subject is an infant.

16. The method of claim 1, wherein the subject is a child.

17. The method of claim 1, wherein the subject is an adult.

18. The method of claim 1, wherein the live, purified population of bacteria is present in a total amount of up to 10\hat{ }15 CFU.

19. The method of claim 1, wherein the live, purified population of bacteria is present in a total amount of at least 10\hat{ }3 CFU.

20. The method of claim 1, wherein the live, purified population of bacteria is present in a total amount of 10\hat{ }3 CFU to 10\hat{ }12 CFU.
